(19) United States
(12) Patent Application Publication
(10) Pub. No.: US 2017/0326309 A1
(43) Pub. Date: Nov. 16, 2017

(54) NEEDLE AND NEEDLE SHIELD ASSEMBLY

(71) Applicant: Owen Mumford Limited, Oxford (GB)

(72) Inventor: Jack Calvert, Oxford (GB)

(21) Appl. No.: 15/533,342

(22) PCT Filed: Dec. 3, 2015
(86) PCT No.: PCT/GB2015/053707

§ 371 Date: Jun. 5, 2017

(30) Foreign Application Priority Data
Dec. 4, 2014 (GB) 1421573.5

(51) Int. Cl. A61M 5/32; A61M 5/24

(52) U.S. Cl. CPC A61M 5/3216; A61M 5/3202; A61M 5/2466; A61M 2005/2474; A61M 2205/581; A61M 2205/582; A61M 2005/3217

(57) ABSTRACT

According to the invention there is provided a needle assembly (1) for mounting on an injection device. The needle assembly comprises; a main body (10) configured to be removably attached to the injection device in use and a needle (90) is secured in the main body (10) having a forward portion which projects forwardly from the main body. A shield (60) is pivotally connected with the body portion by a hinge portion (80). The shield (80) is rotatably movable between: a first position, in which the forward portion of the needle (90) is exposed for use, and a second position, in which the shield (60) is positioned over the forward portion of the needle (90) such that the forward portion of the needle is substantially enclosed within the shield. The shield (60) comprises a pair of spaced apart side walls (64, 66). The base of the side walls comprise a first cooperating feature (72, 74) for engaging a second cooperating feature (22, 24) of the main body (10). The cooperating features are configured to engage to hold the shield in its second position independently of the hinge.

### FIELD OF THE INVENTION

This invention relates to needle assemblies for attachment to a syringe or cartridge arrangement or other injection device.

### BACKGROUND OF THE INVENTION

There are many situations in which an injection needle is required to be shielded after use to prevent the risk of needle stick injury. This applies not only to single use devices such as disposable syringes and the like, but also to multiple use devices such as cartridges where a disposable needle is secured, e.g. by screwing or other suitable connection action, into a cartridge or adaptor and replaced for each subsequent injection. Such needles may be referred to as “pen needles”. The shielding of such needles is especially important in clinics and hospitals where the user is injected by a clinician, where the risk of cross-infection is greater.

WO2014/003632 discloses a protective cover for use with disposable injection needles, such as those used with insulin pens. The protective cover includes a base which is integrally formed with a needle cover; and a needle which is fastened in the base. The needle cover is pivotably attached to a rear portion of the base. After injection, the needle cover is pivoted into an active position in front of the needle, and the action of bringing the hood into the active position bends the needle so that it cannot be reused.

A potential problem with this arrangement is that the user must apply force to the cover in order to first push the cover past the shoulders on the base; and then to bend the needle to bring the cover into the active position. This may be particularly difficult for users with limited movement or strength in their hands and/or fingers. Another problem is that the cover is joined to the base by two integrally formed hinge elements. The hinge elements are thin sections of plastic which means they may be weak and easily snapped. This means that there is a risk that the cover may become partially of fully detached from the base and the cover will no longer function and lock properly. Further, the configuration described may be difficult to form as a single moulded piece.

Other needle shield arrangements are also known which are biased towards a position in which the needle is covered such that the needle only projects from the shield during the delivery of the injection with the shield automatically moving back into a shielding position as the needle is removed from the skin. However, such arrangements may add significant complexity and cost to a needle assembly (particularly a single use, disposable needle assembly). Further with such devices the user is generally unable to see the needle during insertion which many users may find disadvantageous.

Embodiments of the invention seek to provide needle shield assemblies which may overcome some or all of these problems.

### SUMMARY OF THE INVENTION

Accordingly, in one aspect, this invention provides a needle assembly for mounting on an injection device, comprising:
a main body configured to be removably attached to the injection device in use ;
a needle secured in the main body and having a forward portion which projects forwardly from the main body; and
a shield pivotally connected with the body portion by a hinge portion provided between the shield and the main body;
wherein the shield is rotatably moveble between:
a first position, in which the forward portion of the needle is exposed for use, and
a second position, in which the shield is positioned over the forward portion of the needle such that the forward portion of the needle is substantially enclosed within the shield; and
wherein the shield comprises a pair of spaced apart side walls, the base of the walls being adjacent to the main body when the shield is in the second position, and wherein the base of the walls comprise a first cooperating feature for engaging a second cooperating feature of the main body,
the first and second cooperating features being configured to engage to hold the shield in its second position independently of the hinge.

According to another aspect, the invention provides a needle assembly for mounting on an injection device, comprising:
a main body configured to be removably attached to the injection device in use;
a needle secured in the main body and having a forward portion which projects forwardly from the main body; and
a shield pivotably connected to the body portion and including a cavity shaped and dimensioned to enclose the needle after use;

wherein the shield is rotatably movable between:

a first position, in which the forward portion of the needle is exposed for use, and

a second position, in which the shield is positioned over the forward portion of the needle such that the forward portion of the needle is substantially enclosed within the shield cavity.

The shield and main body may be provided with first and second cooperating features which are configured to engage to hold the shield in its second position.

The first and second cooperating features may be configured to provide an audible and/or tactile indication as they engage.

The first cooperating feature may be at least one locking tab. The at least one locking tab may be provided on one of the shield and the main body. The second cooperating feature may comprise at least one slot provided in the other of the shield and the main body. Each slot may be configured to receive a locking tab. The tab may be arranged to resiliently engage the slot.

The shield may comprise a pair of spaced apart side walls. Cooperating features may be formed on at least one of the side walls on a portion of the side wall which is adjacent to the main body when the shield is in the second position. Cooperating features may be provided on each of the two side walls, on a portion of the side wall which is adjacent to the main body when the shield is in the second position.

The cooperating feature may extend from the base of each wall. The cooperating feature may extend inwardly from the base of each wall. The cooperating features may extend outwardly from the base of each wall. The cooperating features may be a latch or tab provided at the base of each wall.

The side walls may be connected by a rear wall. The rear wall may have an outer profile provided with gripping means. The outer surface of the rear wall which is adjacent to the main body when the shield is in the first position may be provided with a profiled or gripping surface.

The shield may be pivotably movable about a point on the main body. The shield may be pivotably movable about a point on a forward portion of the main body. The shield may be pivotably movable about a point on a forward peripheral edge of the main body. The shield portion may be pivotally connected with the main body by a hinge.

The hinge may comprise first and second interconnecting or cooperating hinge elements formed on the shield and the main body. The hinge may include first and second interconnecting or cooperating hinge elements. The hinge elements may for example be arranged to snap fit together to form a hinge in use. The main body may be provided with an integrally formed hinge element. The shield portion may be provided with an integrally formed hinge element, being configured to cooperate with the main body hinge element to form the hinge portion

The hinge may be a separate component which is attached or mounted onto the shield and the main body.

The shield portion may be integrally formed with the main body. The shield portion may be integrally moulded with the main body. The shield portion and the main body may be formed as individual moulded component.

A flexible portion may be provided between the shield and the main body. The flexible portion may be provided integrally formed with both the shield and the main body. The flexible portion may be a living hinge.

The shield may be provided on a forward portion of the main body. The flexible portion may substantially extend along a circumferential section between an upper portion of the body and the shield. The flexible portion (or hinge) may extend substantially tangentially to the forward periphery of the main body. The flexible portion may be formed along a line which is substantially tangential to the forward peripheral edge of the main body.

The interface surface between the forward surface of the main body and the forward surface of the shield may be a sloped surface, the surface being sloped in a plane at an oblique angle to the longitudinally extending needle. The sloped forward surface of the shield may extend substantially continuously from the main body. The sloped forward surface of the shield may extend substantially along the same plane as the sloped forward surface of the main body. The forward surface of the main body may slope from the cooperating features to the hinge, such that the pivot arm is extended in comparison to the perpendicular.

The cooperating features may be configured to retain the shield in the second position independently of the flexible portion.

The needle shield may be biased towards the first (or open) position. The shield may be provided with a flexible connection or living hinge. The flexible connection (or living hinge) may be configured to bias the shield towards its first or open position.

The needle shield may be biased towards the second (or closed) position. The needle shield may be provided with a flexible connection or living hinge, which is configured to bias the shield towards its second or closed position. The needle assembly may further comprise attachment means to hold the shield in the first position. The attachment means may be configured to hold the shield in its first position. The attachment means may include a first engagement feature provided on an outer surface of the main body on the side adjacent to shield. The first engagement feature may be provided on an outer, rearward facing surface of the shield. The engagement feature may be, for example, a latch or hook. The attachment means may include first and second engagement features provided on the shield and the main body, configured to engage each other.

The main body and shield portion may be formed as a single integrally moulded piece.

The needle may be double ended with a rear end opposite the forward end, the rear end being configured in use for piercing a seal element. Such an arrangement may be used in a needle to be attached to an injection device (such as an autoinjector) as the rear needle portion may be configured to pierce the septum of a cartridge or syringe when attached to the injection device.

The needle assembly may further comprise a removable protector which shields or covers the forward end of the needle, and may also shield or cover at least a part of the forward portion of the main body. The needle assembly can be provided with a pre-injection protector, such as a container, needle shield or sheath, or any other well-known form of protection, which protects the user from accidental harm when handling the needle assembly before injection. The protector can be provided to shield or cover the forward end of the needle, and it may also shield or cover the forward portion of the needle assembly. The removable connector may provide a sterile seal to the needle assembly prior to use.

According to another aspect, the invention provides a needle assembly for mounting on an injection device, comprising:
a main body configured to be removably attached to the injection device in use ;
a needle secured in the main body and having a forward portion which projects forwardly from the main body; and
a shield pivotally connected with the body portion;
a hinge portion provided between the shield and the main body;

wherein the shield is rotatably movable between:
a first position, in which the forward portion of the needle is exposed for use, and
a second position, in which the shield is positioned over the forward portion of the needle such that the forward portion of the needle is substantially enclosed within the shield; and

wherein the shield and main body are provided with first and second cooperating features which are configured to engage to hold the shield in its second position independently of the hinge portion.

According to another aspect, the invention provides a needle assembly for mounting on an injection device, comprising:
a main body configured to be removably attached to the injection device in use ;
a needle secured in the main body and having a forward portion which projects forwardly from the main body; and
a shield pivotally connected with the body portion
a hinge portion provided between the shield and the main body at forward edge of the main body;

wherein the shield is rotatably movable between:
a first position, in which the forward portion of the needle is exposed for use, and
a second position, in which the shield is positioned over the forward portion of the needle such that the forward portion of the needle is substantially enclosed within the shield.

The main body and shield portion may be formed as a single integrally moulded piece. The main body and the shield portion may be formed individually as moulded pieces.

The hinge portion may include first and second interconnecting or cooperating hinge elements. The main body may be provided with an integrally formed hinge element. The shield portion may be provided with an integrally formed hinge element, being configured to cooperate with the main body hinge element to form the hinge portion.

The shield and/or main body may be formed from a suitable plastic material, typically a polymer, such as but not limited to propylene.

The main body may include a first body component and a second body component. For example, the main body may comprise a substantially standard needle fixed in a hub, the hub including a threaded section for connection to the injection device; and a second over moulded component. For example the first component may be a substantially standard pen needle. The second component may for example be formed over or attached to the pen needle in an additional manufacturing step. The first body component may comprise a connection mechanism for connecting the needle assembly to a syringe, cartridge or other injection device. The first (rear) body component may be mounted to or on the rear of the second (forward) body component. The first body component may be mounted inside the second body component. The second body component may have a rearwardly facing recess or cavity; and a bore or cavity extending forwardly from the recess to the forward surface of second body component. The first body component may be mounted in the recess of the second body component.

The first body component may include a needle bore. The second body component may include a needle bore which aligns with the needle bore in the first body component when the two components are assembled.

The needle may be mounted in or secured to the second body component, such that when the second body component is mounted in or to the first body component, the forward end of needle extends forwardly through the first body component. The second body component may be releasably mounted in or to the first body component. The second body component may be non-removably secured in or to the first body component, for example by gluing.

The rear portion of the main body may comprise a connection mechanism for connecting the needle assembly to a syringe, cartridge or other injection device. The connection mechanism may be a threaded portion, bayonet fitting or any other suitable engagement element which corresponds to the engagement element on the injection device.

The rear portion of the main body may be configured to be attached to standard hubs provided on the front of syringes, cartridge arrangements or other injection devices. This means that the needle assembly can be attached to any of a range of standard injection devices.

The needle assembly of the invention is a disposable, single-use, needle assembly for use with an injection device. The injection device may be a multi-use or reusable device. In a multi-use injection device, the syringe/cartridge may be replaceable or disposable, and/or arranged to deliver a plurality of doses.

The needle assembly may be a pen needle assembly for use with a pen-type injector.

To assemble the needle assembly, the needle may be vertically mounted into the main body, along the longitudinal needle axis and secured in position.

If the main body includes two components, the needle may be vertically assembled or mounted into the first body component and secured against longitudinal movement, for example by gluing. The first body component may be vertically mounted into and secured in the rear recess of the second body component.

The needle assembly may be mounted or attached to the forward end of an injection device, by screwing or other suitable connection action.Whilst the invention has been described above, it extends to any inventive combination of the features set out in the following description or claims or drawings.

### BRIEF DESCRIPTION OF THE DRAWINGS

FIG. 1 shows a perspective view of a first needle assembly in accordance with an embodiment of the invention in a first configuration, with the needle in an exposed condition ready to deliver an injection;

FIG. 2 shows a side view of the needle assembly of FIG. 1;

FIG. 3 shows a cross-sectional view through line B-B of the retractable needle assembly of FIG. 2;

FIG. 4 shows an end view of the needle assembly of FIG. 1;

FIG. 5 shows the first needle assembly in a second configuration, with the needle shielded;

FIG. 6 shows a side view of the needle assembly of FIG. 5;

FIG. 7 shows a cross-sectional view through line C-C of the needle assembly of FIG. 6;

FIG. 8 shows an end view of the needle assembly of FIG. 5;

FIG. 9 shows a perspective view of a second needle assembly in accordance with another embodiment of the invention in a first configuration with the needle in an exposed condition; and

FIGS. 10A and 10B show a perspective view of the second needle assembly in a second configuration, with the needle shielded.

The embodiments illustrated in the Figures are needle assemblies intended to be screwed or otherwise attached to the forward end of a syringe, cartridge or injection device to allow injection of a substance.

### DESCRIPTION OF AN EMBODIMENT

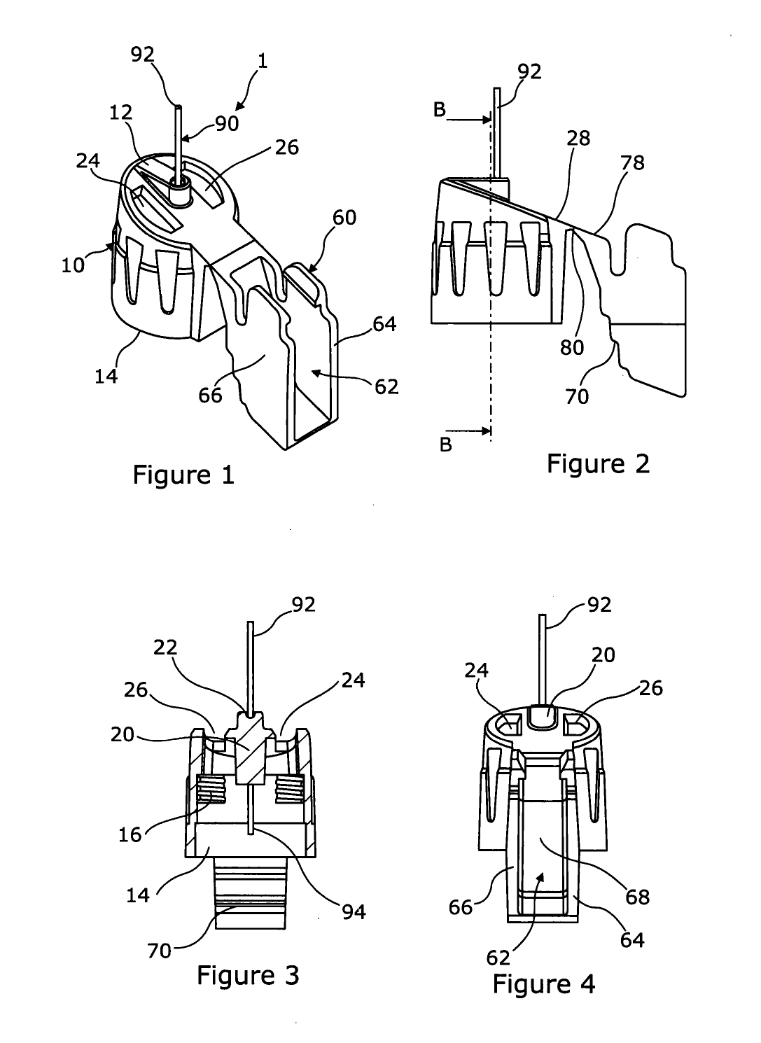

In the following embodiments, the terms “forward” and “front” refer to the patient end of the needle assembly or component thereof, and the term “rear” refers to the non-patient end of the needle assembly or component thereof. In other words, the term “rear” means distant or remote from the injection site during use. FIG. 1 shows a needle assembly 1 in a first configuration, which is the configuration of the assembly before and during injection. The needle assembly 1 includes a main body 10, a needle 90, and a shield 60.

The main body 10 has a front portion 12 and a rear portion 14. The rear portion 14 includes a rear cavity with a threaded portion 16 for attaching the needle assembly to the front end of a syringe, cartridge or other injection device. The main body 10 includes a central portion 20 having a needle bore 22 extending through it (FIG. 3). The needle 90 extends longitudinally along a needle axis. An intermediate portion of the needle 90 is secured in the central portion 20; a forward end 92 of the needle extends forwardly from the needle bore 22 and a rear end 94 of the needle extends rearwardly in the rear portion 14.

A slot 24, 26 is provided each side of the central portion on a forward facing surface of the main body 10.

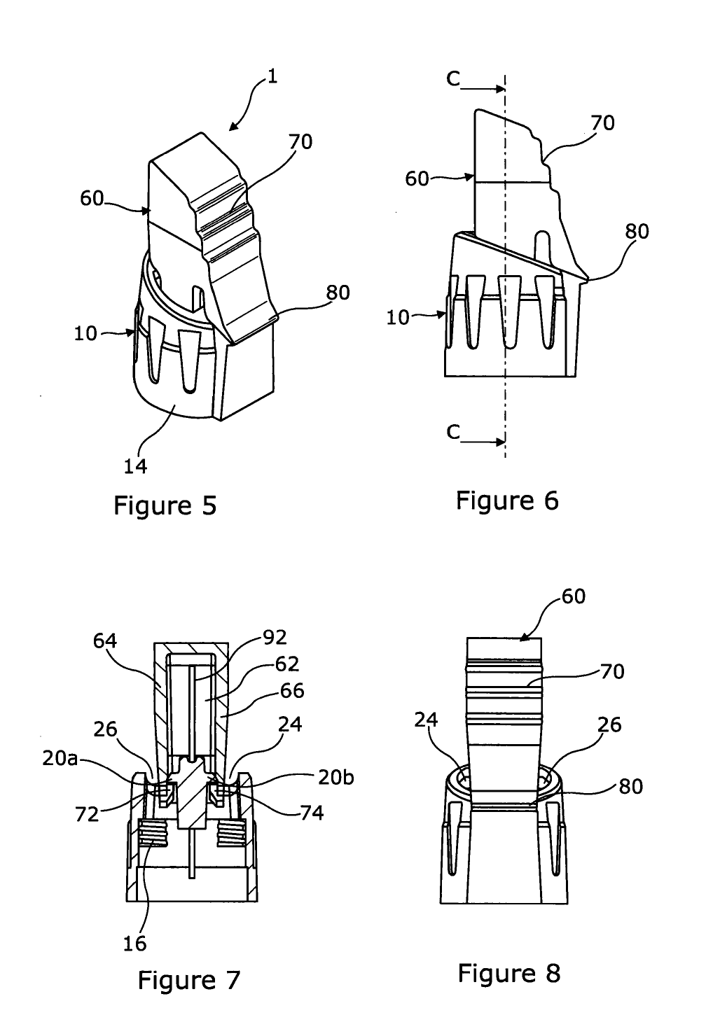

A shield portion 60 is integrally formed with the main body 10 via a living hinge 80. In FIGS. 1-4, the shield 60 is in a first, open position. The hinge 80 is a butterfly-type hinge, although any type of hinge may be used. The hinge line 80 is formed along a line which is substantially tangential to the forward peripheral edge of the main body 10. The shield 80 can be pivoted about the hinge to move it from the first position (FIG. 1) to a second, closed position (FIG. 5).

As can be seen in FIG. 2, in this embodiment, the forward facing or front surface 28 of the main body 10 is a sloped surface, or in other words it extends towards the hinge 80 in a plane at an oblique angle to the needle axis. The forward surface 78 of the shield 60 extends substantially continuously from the main body, along the same plane as the sloped forward surface 28 of the main body (FIG. 2).

The shield 60 includes a cavity 62 (FIGS. 1 and 4) which is defined by side walls 64, 66 and a rear wall 68 extending between the side walls. The cavity 62 is open towards the forward direction (the forward face) and in a direction away from the main body (the outer face). In other embodiments, not shown, the cavity is partially open towards the forward direction and in a direction away from the main body. In other words, the forward face and/or the outer face of the cavity are not completely open. For example, the forward face may include a wall having a central opening, the opening being configured to allow the needle to move into the cavity as the shield moves into the second position.

The forward ends, or bases, of the side walls 64, 66 are provided with latches or tabs 72, 74 which extending inwardly into the cavity 62. In other embodiments, not shown, the latches extend outwardly.

The shield 60 is provided with an ergonomic profile, for example a grip surface 70, on the non-patient or rear surface of the rear wall 68, thus, the surface may be clearly distinguishable as the surface on which the user pushes to move the shield 60.

In FIGS. 5-8, the needle assembly 1 is in its second configuration in which the front end of the needle 92 is covered or shielded. The shield 60 has been pivoted about the hinge 80 to the second position, in which the forwardly projecting end of the needle 92 is entirely located inside the cavity 62. The latches 72, 74 protrude into the slots 24, 26 on the front surface 28 of the main body (FIG. 7) and resiliently engage with protrusions 20a, 20b which extend outwardly from the central portion 20. This locks the shield 60 in its second position.

As can be seen from FIG. 7, the needle 90 remains in the same orientation when the shield is moved into the second position. The needle remains extending longitudinally along the needle axis and it is undeformed.

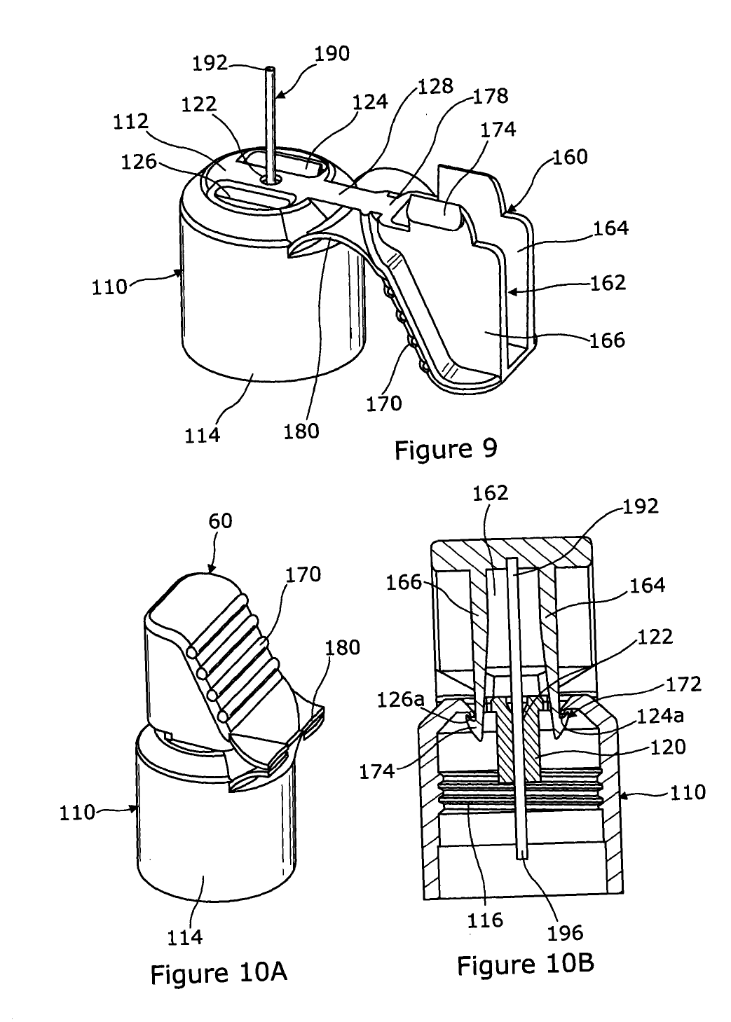

FIG. 9 shows a second needle assembly 1 in a first configuration, which is the configuration of the assembly before and during injection. The needle assembly 101 includes a main body 110, a needle 190, and a shield 160.

The main body 110 has a front portion 112 and a rear portion 114 having a threaded portion 116. The main body 110 includes a central portion 120 having a needle bore 122 extending through it (FIG. 10B). Two slots 124, 26 are provided on a forward facing surface 128 of the main body, one each side of the central bore 122.

A shield portion 160 is integrally formed with the main body 110 via a living hinge 180. In this embodiment, the forward facing surface 128 of the main body 110 is substantially perpendicular to the needle axis. The forward surface 178 of the shield 160 extends continuously with the forward surface 128, along the same plane as the forward surface 128. The shield 160 includes a cavity 162 (FIG. 9), which is open towards the forward direction, and is defined by side walls 164, 166 and a rear wall 168. In this embodiment the latches 172, 174 extend outwardly. In other embodiments, not shown, the latches may extend inwardly into the cavity.

FIGS. 10A and 10B show the needle assembly in the second, closed or safe configuration. The shield 160 has been pivoted into to its second position. The latches 172, 174 engage with projections 124a, 126a which extend rearwardly from outer edges of the slots 124, 126 (FIG. 10B). This locks the shield 160 in its second position.

In use, the needle assembly 1, 101 is attached on to the front end of an injection device (not shown). The shield remains attached to the injection device, in the first or open position when it is mounted or attached to an injection device, and during operation of the injection device. Since the needle shield is provided integrally with and securely attached to the main body, it will not easily become detached and lost. The forward of the needle is fully exposed during the injection providing visual confirmation of the injection to the user. After the injection is complete, the user can simply press on the grip features with a thumb or other digit to pivot the needle shield 60, 160 to its second position. Alternatively, the user can press the grip features against a table or other surface. Only limited force is required by the user to rotate the shield. As the shield 60, 160 is pushed into its second position, the latches 72 and 74, 172 and 174 positively engage in the slots 24 and 26, 124 and 126. The user can feel the positive engagement and hear a clicking noise. This provides a positive indication that the needle assembly is securely held in the second, safe configuration. The user can then safely remove the needle assembly from the injection device and disposed of it without any risk of needle-stick injury.

Since the shield is held securely in the second position, the needle assembly is essentially locked against further opening. This prevents the needle from being used again and reduces the risk of infection and injury due to blunting of the needle which results from multiple uses. As the latches are configured to independently retain the shield in the second position the integrity of the hinge is not essential to the safety state of the needle assembly after use.

In the above embodiments of the invention, the needle assembly is formed as a single moulded plastic component. The configuration of the invention is simple and easy to manufacture and assemble, which means the cost is low. This is important for single use or disposable needle assemblies.

In any embodiments of the invention, the needle assembly may also be provided with needle cover or sheath (not shown) which covers the forward end of the needle prior to use. The needle assembly is provided ready for use with the needle cover mounted over the forward end of the needle. This protects the user from needle-stick injury when attaching the needle assembly to the injection device.

In other embodiments (not shown), the shield portion is connected to the main body by a hinge. The main body is provided with an integrally formed hinge element. The shield portion is provided with an integrally formed hinge element, the shield hinge element being configured to cooperate with the main body hinge element to form the hinge.

In other embodiments (not shown) the main body is made of up two components. A first body component is a substantially standard needle hub. The rear portion of hub includes a threaded section for connection to the injection device. The intermediate portion of the needle is secured, for example by gluing, into the hub. The main body also includes a second forward component having a recess into which the hub is mounted. A needle bore extends centrally in the second component from the recess to a forward surface, so that when the hub and needle are mounted in the recess, the forward portion of the needle extends forwardly from the main body. The shield is formed integrally with the main body in the same way as described in the previous embodiments.

The needle assembly may also be provided with a further indicator (not shown) which provides at least one of a tactile, an audible and a visual indication that the shield is secured to the main body portion.

1. A needle assembly for an injection device, comprising:
a main body configured to be removably attached to the injection device when in use;
a needle secured in the main body and having a forward portion which projects forwardly from the main body; and
a shield pivotally connected with the main body by a hinge portion provided between the shield and the main body,
wherein the shield is rotatably movable between:
a first position, in which the forward portion of the needle is exposed for use, and
a second position, in which the shield is positioned over the forward portion of the needle such that the forward portion of the needle is substantially enclosed within the shield, and
wherein the shield comprises a pair of spaced apart side walls, a base of the walls being adjacent to the main body when the shield is in the second position, and wherein the base of the walls comprise a first cooperating feature for engaging a second cooperating feature of the main body, and
wherein the first and second cooperating features are configured to engage to hold the shield in the second position independently of the hinge.

2. The needle assembly according to claim 1, wherein the first cooperating feature comprises tabs provided on one of the shield and the main body, and the second cooperating feature comprises slots provided in the other of the shield and the main body, and
wherein the slots are configured to receive the locking tabs and the tabs are arranged to resiliently engage the slots.

3. The needle assembly according to claim 2, wherein the shield portion is integrally formed with the main body.

4. The needle assembly according to claim 1, wherein the hinge is an integrally formed flexible portion provided between the shield and the main body.

5. The needle assembly according to claim 1, wherein the shield is provided on a forward portion of the main body.

6. The needle assembly according to claim 5, wherein the hinge substantially extends along a circumferential section between an upper portion of the body and the shield.

7. The needle assembly according to claim5, wherein the hinge extends substantially tangentially to a forward periphery of the main body.

8. The needle assembly according to claim 7, wherein an interface surface between the forward periphery of the main body and a forward surface of the shield is a sloped surface.

9. The needle assembly according to claim 1, wherein the cooperating features extend from the base of each of the walls.

10. The needle assembly according to claim 1, wherein the pair of walls are side walls, which are connected by a rear wall, the rear wall having an outer profile provided with gripping means.

11. The needle assembly according to claim 1, wherein the first and second cooperating features are configured to provide an audible and/or tactile indication as they engage.

12. (canceled)

13. (canceled)

14. A needle assembly comprising:
a main body;
a needle secured with the main body and having a forward portion that projects from the main body; and
a shield pivotally connected with the main body by a hinge, the shield including a pair of spaced apart side walls, and a base of the walls including a first cooperating feature for engaging a second cooperating feature of the main body,
wherein the shield is movable between:
a first position, in which the forward portion of the needle is exposed for use, and
a second position, in which the shield is positioned over the forward portion of the needle such that the forward portion of the needle is substantially enclosed within the shield,
wherein the base of the walls is adjacent to the main body when the shield is in the second position, and
wherein the first and second cooperating features are configured to engage to hold the shield in the second position independently of the hinge.

15. The needle assembly according to claim 14, wherein the hinge is a butterfly hinge.

16. The needle assembly according to claim 14, wherein the hinge is an integrally formed flexible portion provided between the shield and the main body.

17. The needle assembly according to claim 14, wherein the first cooperating feature comprises tabs provided on one of the shield and the main body, and the second cooperating feature comprises slots provided in the other of the shield and the main body.

18. The needle assembly according to claim 14, wherein an interface surface between a forward surface of the main body and a forward surface of the shield is a sloped surface.

19. The needle assembly according to claim 14, wherein the cooperating features extend from the base of each of the walls.

20. The needle assembly according to claim 14, wherein the pair of walls are connected by a rear wall, the rear wall having an outer profile provided with gripping means.

21. The needle assembly according to claim 14, wherein the first and second cooperating features are configured to provide an audible or tactile indication as they engage.

22. The needle assembly according to claim 14, wherein when the shield is in the second position, the needle is completely covered by the shield.
